(19) United States
(12) Patent Application Publication
(10) Pub. No.: US 2026/0306220 A1
(43) Pub. Date: Oct. 8, 2026

(54) MORPHOGENIC BONE ACCELERATOR

(71) Applicant: Mauricio Gonzalez Balut, San Bernardino, CA (US)

(72) Inventor: Mauricio Gonzalez Balut, San Bernardino, CA (US)

(21) Appl. No.: 19/482,185

(22) PCT Filed: Jan. 25, 2023
(86) PCT No.: PCT/US2023/011541

§ 371 Date: Nov. 6, 2025

(51) Int. Cl. A61C 7/08

(52) U.S. Cl. CPC A61C 7/08

(57) ABSTRACT

Disclosed herein are novel methods, devices, and systems for facilitating and/or accelerating movement and/or alignment of teeth by using radiofrequencies such as X-rays. Specifically disclosed is a morphogenetic bone accelerator (MBA) comprising two segments, an upper segment that is configured to cover the upper arch (or maxilla) of a patient's mouth, and a lower segment that is configured to cover the lower arch (or mandible) of the patient's mouth. Both the upper and the lower segments of the MBA comprise a metal wire surrounded by a soft body. The wire conducts or translates benign radiation emitted from a radiation emitting device into a patient's mouth. Such radiation may be low-energy surface X-rays (e.g., having a radiation power range of between 10 and 100 kilovolts (kV)) and/or medium and deep X-rays (e.g., having a radiation power range of between 120 and 200 kV).

### FIELD OF THE DISCLOSURE

The disclosure relates generally to novel medical orthodontic devices, systems, and/or methods for facilitating and/or accelerating movement and alignment of teeth. In particular, embodiments of the disclosure relate to a morphogenetic bone accelerator (MBA) device for accelerated orthodontic treatment, where the device emanates benign, controlled, and localized radiotherapy and/or radiofrequencies, thereby resulting in accelerated movement and alignment of teeth without damaging adjacent tissues and structures.

### BACKGROUND

Orthodontic treatment in many individuals can be time-consuming and painful due to the need to gradually move teeth through gum tissue so that they are properly aligned.

Current state-of-the art devices, systems, and methods for facilitating the movement and/or alignment of teeth rely on physical force applied to one or more teeth (e.g., retainers) or non-specific and unproven pulses or vibrations. For example, retainers are generally known in the art as rigid devices, often made of metal or clear plastic, that rely on physical force to hold, move, and/or align teeth into position. Removable retainers such as the Hawley retainer generally use a metal wire to surround the six anterior teeth and keep them in place. The metal wire can be anchored in a baseplate made of, for instance, acrylic. One of skill in the art will recognize many different retainer variations are possible, including, for example, circumferential retainers that consist of a wrap-around labial bow and no clasps. Such retainers can interfere with speech and risk breaking of fracturing.

However, such retainers can interfere with speech, risk breaking or fracturing, and are ill-suited to prevent incisor irregularity relapse.

Given the foregoing, there exists a significant need for improved methods, devices, and or systems for facilitating the movement and/or alignment of teeth as part of orthodontic treatment.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In certain embodiments, the disclosed embodiments may include one or more of the features described herein.

Embodiments of the present disclosure are directed towards novel medical orthodontic devices, systems, and/or methods for facilitating and/or accelerating movement and alignment of teeth using controlled and localized radiotherapy and/or radiofrequencies.

In at least one embodiment, a novel morphogenetic bone accelerator (MBA) is disclosed. The MBA is a device that emanates benign radiotherapy and/or radiofrequencies for accelerated orthodontic treatment. Such radiotherapy and/or radiofrequencies stimulate bone remodeling and accelerate teeth movement without surgery or any invasive procedure and without damaging adjacent tissues and/or anatomical structures.

The MBA may, in at least one embodiment, comprise two segments, an upper segment that is configured to cover the upper arch (or maxilla) of a patient's mouth, and a lower segment that is configured to cover the lower arch (or mandible) of the patient's mouth. Both the upper and the lower segments of the MBA comprise a metal wire or other material suitable for emitting radiofrequency (RF) energy, and may be surrounded by a soft body. The wire may be made of a metal that is flexible and/or bendable, as well as electrically conductive (e.g., nitinol). The soft body may be made of a material that is soft, spongy, and moldable around the wire, such as polyethylene foam. The wire, as well as the soft body, may be made in different sizes so that the MBA can adapt to the exact size and shape of the patient's mouth.

In use, the MBA is placed inside the mouth so that the wire extends around the buccal surfaces above and below the upper and lower teeth, respectively. When inside the patient's mouth, the upper segment of the MBA touches the outer gum tissue on the upper half of the mouth, as well as the palate behind the upper teeth, and the lower segment of the MBA surrounds the gum tissue on the lower half of the mouth. A portion of the wire extends outside of the patient's mouth and is connected to a radiation emitting device. Benign radiation can then be transmitted from the radiation emitting device through the wire, causing the jawbones of the patient to temporarily soften so that the teeth can be moved more easily.

Movement of the teeth during radiotherapy can be accomplished, for example, by the use of traditional orthodontic brackets and archwires. However, unlike with standard orthodontic procedures, all the movement of the teeth may take place in a short period of time (for example 20-30 minutes), without changing the archwires. Accordingly, it may be desirable to prescribe custom orthodontic brackets with slots having an appropriate orientation/configuration to achieve the desired movement of the teeth in conjunction with the archwires that fit into the brackets' slots. Customization of orthodontic brackets is known in the art. Design of such customized brackets may be carried out by taking a scan of the patient's mouth and jaw, so that the exact final placement of the teeth can be decided and brackets prescribed accordingly.

Once the teeth are moved, the MBA and orthodontic brackets/archwires can be removed and the patient can be provided with orthodontic retainers to maintain the shape and straightness of the teeth.

The MBA may use low-energy surface X-rays with a radiation power range of, for example, 10 to 100 kilovolts (kV). In at least one embodiment, the MBA may be tuned to apply a second stage of orthovoltage X-ray therapy, specifically, medium and deep orthovoltage therapy with a radiation power range of, for example, 120 to 200 kV. The X-Rays and/or other RF energy may be generated by an RF generator connected to the MBA. The connection between the RF generator and the MBA may be for example one or more wires.

Thus, embodiments of the disclosure utilize X-rays and X-ray therapy as an alternative to painful and time-consuming surgical procedures.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows a top view of an upper segment of a morphogenetic bone accelerator (MBA) device, according to at least one embodiment of the present disclosure.

FIG. 2 shows a top view of a lower segment of a MBA device, according to at least one embodiment of the present disclosure.

FIG. 3 shows a bottom view of an upper segment of a MBA device surrounding teeth of the upper jaw, according to at least one embodiment of the present disclosure.

FIG. 4 shows a top view of a lower segment of a MBA device surrounding teeth of the lower jaw, according to at least one embodiment of the present disclosure.

FIG. 5 shows both an upper segment and a lower segment of a MBA device, according to at least one embodiment of the present disclosure.

FIG. 6 shows both an upper segment and a lower segment of a MBA device with the upper segment surrounding teeth of the upper jaw and the lower segment surrounding teeth of the lower jaw, according to at least one embodiment of the present disclosure.

FIG. 7 shows a lateral right-side view of a MBA device when placed in the mouth, according to at least one embodiment of the present disclosure.

FIG. 8 shows a lateral left-side view of a MBA device when placed in the mouth, according to at least one embodiment of the present disclosure.

FIG. 9 shows a lingual view of a MBA device when placed in the mouth, according to at least one embodiment of the present disclosure.

FIG. 10 shows an anterior view of a MBA device when placed in the mouth, according to at least one embodiment of the present disclosure.

FIG. 11 shows a lateral right-side view of a MBA device, according to at least one embodiment of the present disclosure.

FIG. 12 shows a lateral left-side view of a MBA device, according to at least one embodiment of the present disclosure.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, the singular forms “a”, “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

As used herein, ranges are used herein in shorthand, so as to avoid having to list and describe each and every value within the range. Any appropriate value within the range can be selected, where appropriate, as the upper value, lower value, or the terminus of the range.

Unless indicated to the contrary, numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

The words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of”. Similarly, the term “consisting essentially of” is intended to include embodiments encompassed by the term “consisting of”. Although having distinct meanings, the terms “comprising”, “having”, “containing’ and “consisting of” may be replaced with one another throughout the description of the invention.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Terms such as, among others, “about,” “approximately,” “approaching,” or “substantially,” mean within an acceptable error for a particular value or numeric indication as determined by one of ordinary skill in the art, which depends in part on how the value is measured or determined. The aforementioned terms, when used with reference to a particular non-zero value or numeric indication, are intended to mean plus or minus 10% of that referenced numeric indication. As an example, the term “about 4” would include a range of 3.6 to 4.4. All numbers expressing dimensions, velocity, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims, each numerical parameter should be construed in light of the number of significant digits and ordinary rounding approaches.

“Typically” or “optionally” means that the subsequently described event or circumstance may or may not occur, and that the description includes instances where said event or circumstance occurs and instances where it does not.

Wherever the phrase “for example,” “such as,” “including” and the like are used herein, the phrase “and without limitation” is understood to follow unless explicitly stated otherwise.

Embodiments of the disclosure comprise novel methods, devices, and/or systems that use radiofrequencies and/or X-rays to facilitate movement and/or alignment of teeth for orthodontic treatment.

In at least one embodiment, a morphogenetic bone accelerator (MBA) is disclosed that emanates and/or emits radiotherapy, in the form of one or more radiofrequencies, to stimulate and accelerate teeth movement. The radiofrequencies are benign, localized, and controlled by the MBA (and/or attached RF-generating machine) such that they do not damage surrounding tissue (e.g., gum tissue) and/or other anatomical structures in the mouth.

The MBA may, in at least one embodiment, comprise two segments, an upper segment that is configured to cover the upper arch (or maxilla) of a patient's mouth, and a lower segment that is configured to cover the lower arch (or mandible) of the patient's mouth. Both the upper and the lower segments of the MBA comprise a metal wire surrounded by a soft body. The wire may be made of a metal that is flexible and/or bendable, as well as electrically conductive (e.g., nitinol). The soft body may be made of a material that is soft, spongy, and moldable around the wire. The wire, as well as the soft body, may be made of different sizes so that the MBA can adapt to the exact size and shape of the patient's mouth.

To adapt the MBA to the mouth size and shape of a given patient, a scan of the mouth may be performed to measure bone presence, structure, and/or density. Then, the MBA can be placed into the patient's mouth such that the upper segment surrounds the gum tissue on the upper half of the mouth, and the lower segment surrounds the gum tissue on the lower half of the mouth. That is, the MBA is positioned so that the wire, surrounded by the soft body, runs around the buccal and/or lingual portions of the gum tissue.

In use, the wire serves to conduct or translate benign radiation from a radiation emitting device into the jawbones of the patient. The soft body covering the wire acts as a cushion and contacts one or more interior surfaces of the mouth such as, for instance, the upper and lower buccal surfaces and the palate.

Orthodontic brackets and archwires may be utilized to move a patient's teeth into the proper position during the radiofrequency treatment. These brackets may be standard orthodontic brackets known in the art.

A portion of the wire may extend outside the patient's mouth and be connected to the aforementioned radiation-emitting device. Benign radiation can then be transmitted from the radiation emitting device through the wire, causing the jawbones of the patient to temporarily soften so that the teeth can be moved as part of an orthodontic treatment.

In at least one embodiment, radiation is sent from the radiation emitting device through the wire, and into the patient's mouth, for 1-2 minutes or less. It should be appreciated that, for patient comfort and/or safety, the type and/or amount of radiation used does not generate or emanate excessive heat into the patient's mouth.

Accordingly, the radiation emitting device may emanate and/or emit low energy surface X-rays with a radiation power range of between 10 to 100 kilovolts (kV). The radiation emitting device can further be tuned and/or altered to apply medium and deep orthovoltage X-rays with a radiation power range of between 120 to 200 kV. A skilled artisan will appreciate that such X-rays are used to treat a variety of different diseases.

The radiation emitting device may further emanate and/or emit direct, controlled, and benign radiotherapy in the form of one or more radiofrequencies having a range of between 50 to 100 kV with a current of between 50 and 100 milliamps (mA). Thus, the range may be, for instance, 50, 75, or 100 kV, and the current may be, for example, 50, 75, or 100 mA. In at least one example, the current may be higher than 100 mA, such as, for example, 120, 140, 150, and even 200 mA.

At least one or more of the MBA devices disclosed herein further have an ergonomic structure that is anatomically fitted and comfortable for the user.

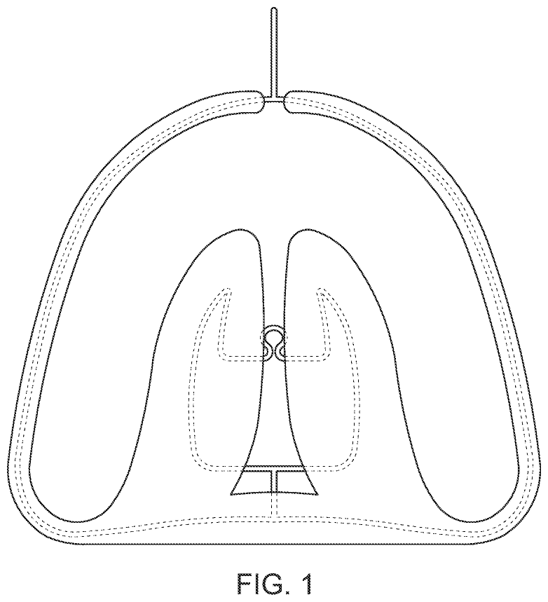

Turning now to FIG. 1, an upper segment of a MBA is shown for use with the upper half of a patient's mouth. As mentioned above herein, the wire is surrounded by a soft body (the pink portion surrounding the inner wire shown), which surrounds the maxilla from the anterior labial segment through to the posterior tuberosity of the maxilla. The MBA further extends to the roof of the upper palate.

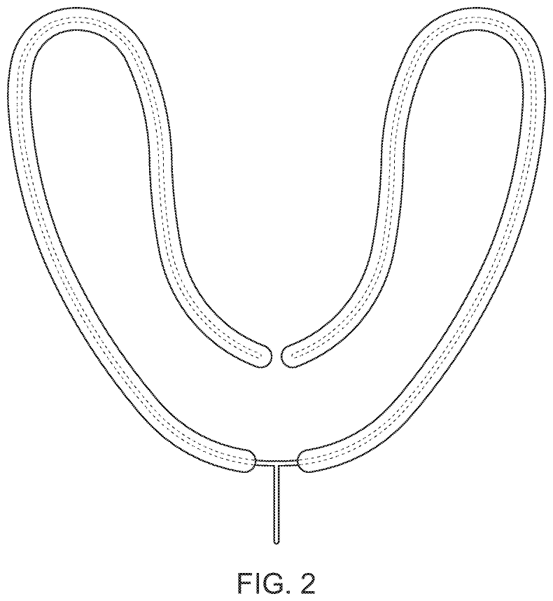

FIG. 2 shows a corresponding lower segment of the MBA for use with the lower half of the patient's mouth. Here, the wire covered by the soft body surrounds the mandible from the anterior labial segment through to the ramus of the mandible, and extends through the lingual surface to the midline.

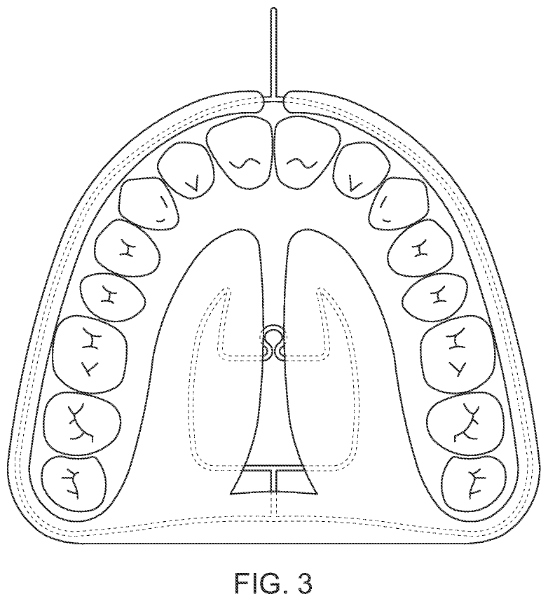

FIG. 3 shows an upper segment of a MBA in use, with the upper dentition shown. The MBA extends from the upper midline of the anterior labial segment through to the posterior tuberosity of the maxilla, and further extends to the roof of the upper palate.

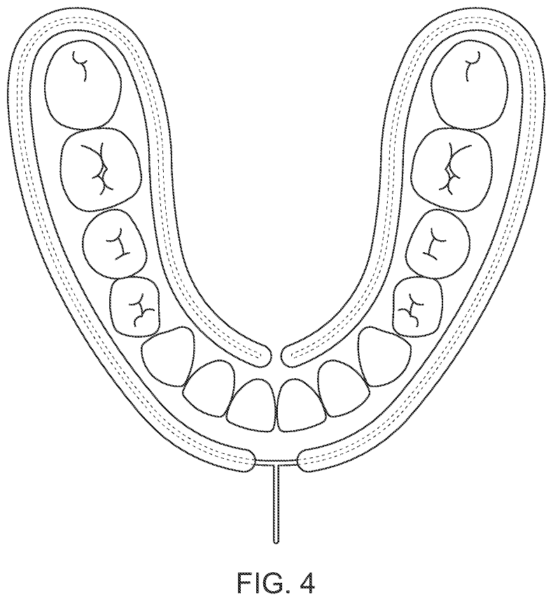

FIG. 4 shows a lower segment of a MBA in use, with the lower dentition shown. The MBA extends from the lower midline of the anterior labial segment through to the ramus of the mandible, and extends through the lingual surface to the midline.

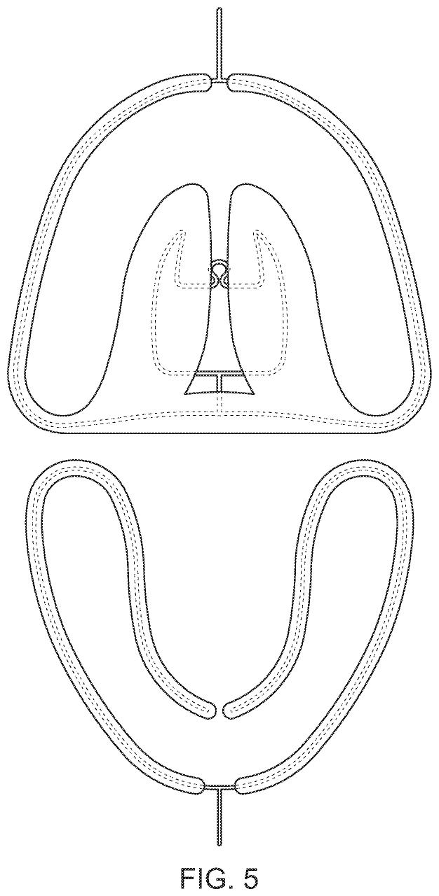

FIG. 5 shows both the upper and the lower segments of a MBA. The upper segment, for use with the upper half of a patient's mouth, extends from the upper midline of the anterior labial segment through to the posterior tuberosity of the maxilla, and further extends to the roof of the upper palate. The lower segment, for use with the lower half of a patient's mouth, extends from the lower midline of the anterior labial segment through to the ramus of the mandible, and extends through the lingual surface to the midline.

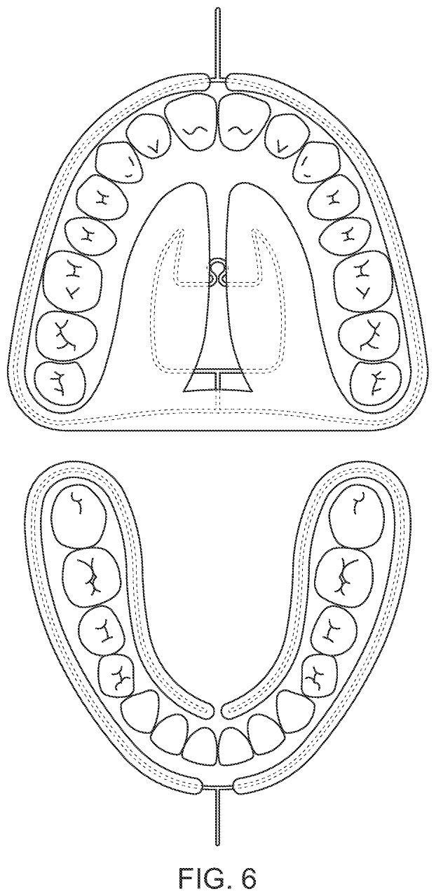

Similarly, FIG. 6 shows both the upper and the lower segments of the MBA of FIG. 5, with both the upper and the lower dentition shown.

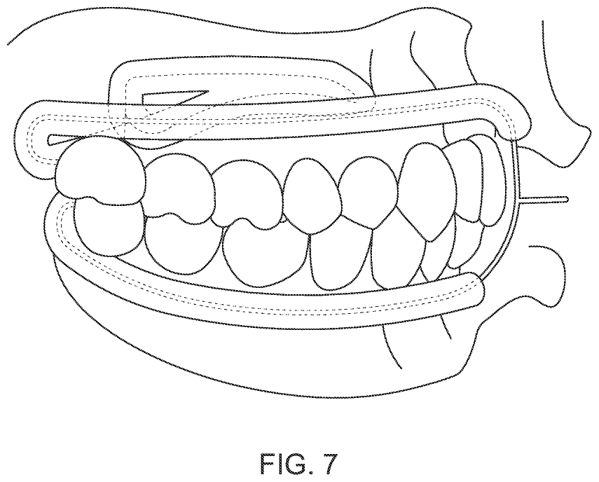

FIG. 7 shows a lateral, right-side view of a MBA, depicting the main midline vastagus uniting the upper segment and the lower segment from the anterior and the labial surfaces, surrounding both the maxilla (for the upper segment) and the mandible (for the lower segment) with the upper and lower dentition, respectively.

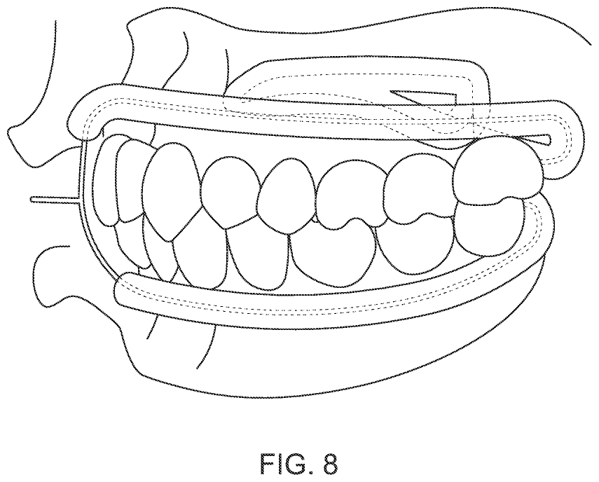

Similarly, FIG. 8 shows a lateral, left-side view of the MBA of FIG. 7.

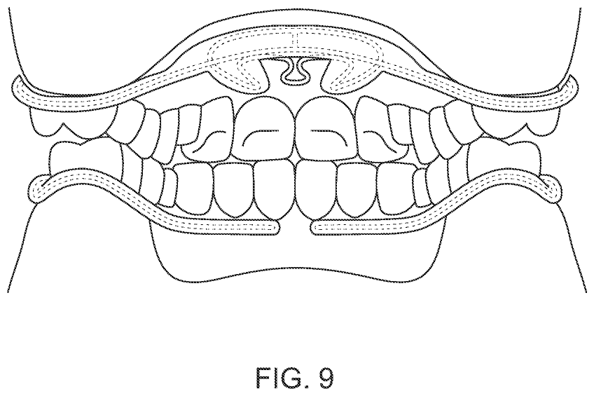

FIG. 9 shows a lingual view of a MBA, depicting the upper and the lower segments from the palatal and lingual surfaces, respectively. The upper segment surrounds the maxilla and upper dentition, through to the posterior tuberosity of the maxilla and through to the roof of the upper palate. The lower segment surrounds the mandible and lower dentition, through to the lingual surface of the mandible.

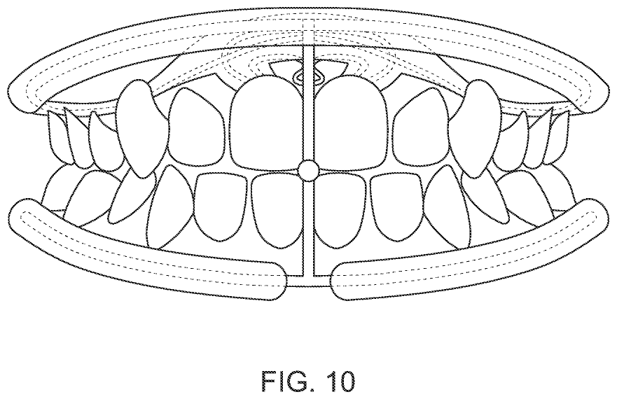

FIG. 10 shows an anterior view of a MBA, depicting the main midline vastagus uniting the upper segment and the lower segment from the anterior and the buccal surfaces, surrounding both the maxilla (for the upper segment) and the mandible (for the lower segment) with the upper and lower dentition, respectively. The upper segment extends through to the posterior tuberosity of the maxilla, and further extends to the roof of the upper palate. The lower segment extends through to the labial surface of the mandible.

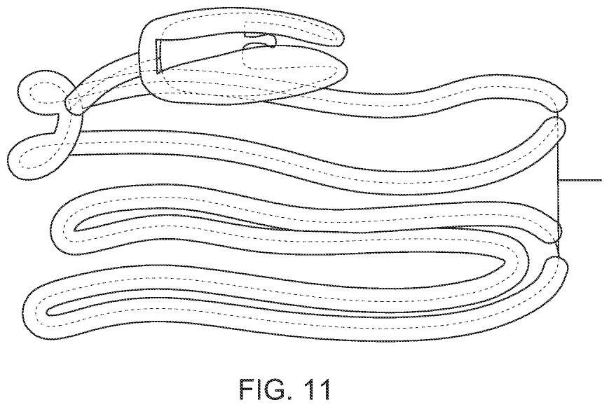

FIG. 11 shows a lateral, right-side view of a MBA, again depicting the main midline vastagus uniting the upper segment and the lower segment from the anterior and the labial surfaces, surrounding both the maxilla (for the upper segment) and the mandible (for the lower segment) with the upper and lower dentition, respectively.

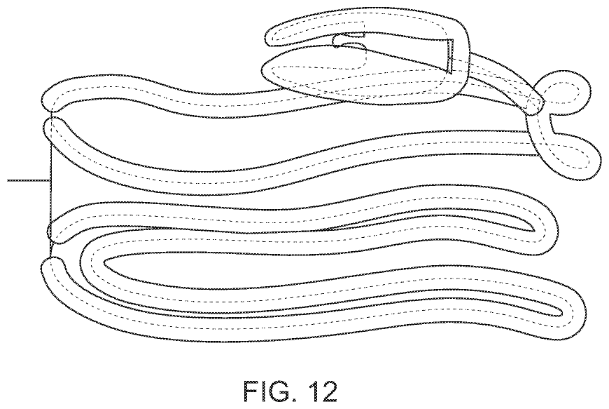

Similarly, FIG. 12 shows a lateral, left-side view of the MBA of FIG. 11.

In at least one embodiment, a method for facilitating movement and/or alignment of teeth is disclosed, the method comprising directing low-energy surface X-rays to a patient's jaw bones(s), wherein the low-energy surface X-rays have a radiation power range of between 10 to 100 kV, and directing medium and deep X-rays to the patient's jaw bone(s), wherein the medium and deep X-Rays have a radiation power range of between 120 to 200 Kv.

In at least a further embodiment, a method for facilitating movement and/or alignment of teeth is disclosed, the method comprising applying benign radiofrequencies inside a patient's mouth, wherein the benign radiofrequencies comprise X-rays. At least ninety percent (90%) of the X-rays are applied horizontally within up to 2.5 millimeters (mm) from the patient's intraoral hard and soft tissues.

Further, the angle of the X-rays can vary in a transverse dimension from between 25 to 90 degrees, and/or in a sagittal dimension from 110 to 180 degrees. The aforementioned results in delivery of required doses of radiation to the bone along tooth roots and gum tissue, thereby enabling accelerated movement of the teeth.

Accordingly, one or more aspects of the MBA device disclosed herein can be manufactured and classed as regulated medical devices, such as, for example, a Class III Medical Device under the U.S. Food and Drug Administration (FDA)'s regulatory classification.

It should be appreciated that the methods, devices, and/or systems disclosed herein, including, for instance, one or more MBA devices described above, can be used for treatment of various malocclusions, accelerating bone remodeling.

It should further be appreciated that the methods, devices, and/or systems disclosed herein, including, for instance, one or more MBA devices described above, can be used for treating various conditions and/or diseases in other areas of the body besides the teeth and mouth, including, for instance, the head, the trunk, and one or more extremities.

Accordingly, embodiments of the disclosure can be used for increased movement and/or alignment of teeth, as well as bone remodeling. Such embodiments are therefore useful for any individuals requiring orthodontic treatment and/or treatment of bone fractures. Indeed, one or more embodiments facilitate orthodontic tooth movement in a time lapse of minutes and the healing of a bone fracture in less than a week.

Therefore, embodiments of the disclosure can be used in a wide variety of consumer and healthcare applications in numerous fields, including, for instance, dentistry, general medicine, the biomedical industry, the medical device industry, and the medical device and/or healthcare industry generally.

These and other objectives and features of the invention are apparent in the disclosure, which includes the above and ongoing written specification.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

I/We claim:

1. A morphogenetic bone accelerator (MBA) comprising:
one or more portions that emit radiation at one or more frequencies on one or more locations inside a patient's mouth,
wherein at least 90% of the radiation is emitted horizontally within 2.5 millimeters from the patient's intraoral hard and soft tissues.

2. The MBA of claim 1, wherein the radiation comprises X-rays having a radiation power range of between 10 and 200 kilovolts (kV).

3. The MBA of claim 2, wherein an angle of the X-rays varies in a transverse dimension from between 25 and 90 degrees, and wherein an angle of the X-rays varies in a sagittal dimension from between 110 and 180 degrees.

4. A method for facilitating movement and/or alignment of teeth, the method comprising:
directing one or more low-energy surface X-rays to a patient's jaw bone; and
directing one or more medium and deep X-rays to the patient's jaw bone,
wherein the one or more low-energy surface X-rays have a radiation power range of between 10 to 100 kilovolts (kV), and
wherein the one or more medium and deep X-rays have a radiation power range of between 120 to 200 kV.
